SEQUENCE LISTING (10 sequences)

SEQ ID NO: 1 - DNA, 20 bp, artificial sequence
  PCR sense primer for CYP 1B1
gtatattgttgaagatacac

SEQ ID NO: 2 - DNA, 20 bp, artificial sequence
  PCR antisense primer for CYP 1B1
aaagaggtacaacatcacct

SEQ ID NO: 3 - DNA, 20 bp, artificial sequence
  PCR sense primer for CYP 2E1
agcacaactctgagatatgg

SEQ ID NO: 4 - DNA, 20 bp, artificial sequence
  PCR antisense primer for CYP 2E1
atagtcactgtacttgaact

SEQ ID NO: 5 - DNA, 20 bp, artificial sequence
  PCR sense primer for CYP 2B6/7
ccatacacagaggcagtcat

SEQ ID NO: 6 - DNA, 20 bp, artificial sequence
  PCR antisense primer for CYP 2B6/7
ggtgtcagatcgatgtcttc

SEQ ID NO: 7 - DNA, 20 bp, artificial sequence
  PCR sense primer for actin
acccacactgtgcccatcta

SEQ ID NO: 8 - DNA, 17 bp, artificial sequence
  PCR antisense primer for actin
cggaaccgctcattgcc

SEQ ID NO: 9 - DNA, 21 bp, artificial sequence
  PCR sense primer for CYP 1A1
tagacactgatctggctgcag

SEQ ID NO: 10 - DNA, 20 bp, artificial sequence
  PCR antisense primer for CYP 1A1
gggaaggctccatcagcatc

(19) United States
(12) Patent Application Publication
(10) Pub. No.: US 2003/0017459 A1
(43) Pub. Date: Jan. 23, 2003

(54) Method for predicting drug clearance and individualized dosage

(76) Inventor: Murali Ramanathan, Amherst, NY (US)

Correspondence Address: Ranjana Kadle, Hodgson Russ LLP, Suite 2000, One M&T Plaza, Buffalo, NY, 14203-2391, US

(21) Appl. No.: 09/891,555

(22) Filed: Jun. 26, 2001

Related U.S. Application Data
(60) Provisional application No. 60/214,189, filed on Jun. 26, 2000.

(51) Int. Cl. C12Q 1/68; C12P 19/34

(52) U.S. Cl. 435/6; 435/91.2

(57) ABSTRACT

The present invention provides a blood based method for estimation of drug metabolizing enzyme RNAs in liver. The method comprises the steps of obtaining a blood sample from an individual, isolating one or more types of cells from the sample, preparing total RNA or mRNA from the cells and subjecting the RNA to DNA arrays comprising probes for desired genes to determine the levels of the mRNAs. These levels are then used to estimate corresponding mRNA levels in liver.

This application claims priority of U.S. provisional application serial No. 60/214,189 filed on Jun. 26, 2000, the disclosure of which is incorporated herein by reference.

This work was supported by a grant from the National Institute of General Medical Sciences no. GM54087. The government has certain rights in the invention.

### FIELD OF THE INVENTION

This invention relates generally to the field of methods for predicting metabolism of drugs. More particularly, this invention provides a method of predicting the clearance of drugs by assaying for nucleic acids related to drug metabolizing enzymes in blood cells.

### BACKGROUND OF THE INVENTION

In pharmacokinetics, drug clearance is a fundamental determinant of dosage. Clearance is a complex phenomenon based on the genotype as well as environmental and physiological factors. Consequently, it shows significant inter-individual variability. Since inappropriate dosage can lead to treatment failures or drug toxicity, the assessment of drug clearance could have significant impact on clinical practice.

Variations in the expression levels of drug metabolizing enzymes and transporters, and genetic polymorphisms are the principal causes of inter-individual and intra-individual variability in drug disposition. Some of the drug-metabolizing enzymes and transporter systems are also responsible for a wide range of drug-drug interactions and for drug resistance.

The metabolism of a drug in the body is a major determinant of clearance (Gibaldi et al., 1982, Pharmacokinetics, M. Dekker, New York). The liver is the predominant site of drug metabolism, although the kidney, gastrointestinal tract, lungs and skin also have significant metabolizing capacities. The cytochrome P450 enzymes (CYPs) are a superfamily of heme-containing proteins that catalyze the oxidation of drugs and xenobiotics in the presence of NADPH, oxygen and a flavoprotein, NADPH-cytochrome P450 reductase. This system is located at the endoplasmic reticulum membrane and is a dominant determinant of metabolism of many drugs. The superfamily of P450 enzymes segregates into several families of genes. The families in turn, are divided into subfamilies whose members share more than 55% amino acid sequence identity. Multiple CYPs can be active in a single tissue and many CYPs may metabolize a single drug. For example, warfarin is metabolized by CYP 2C9, CYP 3A4, CYP 2C19 and CYP 1A2. The importance of the CYPs in overall metabolism can be judged from the fact that human microsomal preparations are widely used in drug screening to assess metabolism, estimate drug hepatic clearance and to provide information on the potential for drug-drug interactions.

Current methods to determine levels or polymorphisms of CYPs involve obtaining microsomal preparations from liver biopsies and therefore are not clinically useful. To overcome the disadvantages of liver metabolism studies, a broad range of blood based methods have been investigated. For the CYP 3A system, for example, tests based on the plasma 1′-hydroxymidazolam to midazolam ratio, 6-hydroxycortisol to cortisol ratio in urine, lidocaine to monethylglycinexylidide ratio and the erythromycin breath test have been investigated. Of these, the erythromycin breath test and midazolam plasma ratio are considered more reliable predictors of CYP 3A (Thummel et al., 1994, J. Pharmacol. Exp. Therap., 271:549-556; Watkins et al., 1989, J. Clin. Invest., 83:688-697), while the reliability of some of the other tests has been questioned (Watkins, 1994, Pharmacogenetics, 4:171-184). However, these methods have not gained wide-spread acceptance because the erythromycin breath test requires administration of [¹⁴C]N-methyl erythromycin and midazolam is considered to cause hypnosis.

There is significant inter-individual variability in CYP levels; the amount of each CYP protein detected can vary by two orders of magnitude. Thus, the currently available data suggest that the CYP system is polymorphic, diverse, broadly specific and variable in expression. Consequently, functional genomics appears to be suitable for the study of such systems.

Several methods have been available for monitoring gene expression or detecting differentiallly expressed genes. Techniques for studying mRNA by comparative hybridization in gels and by differential display have been used for number of years; however they are both cumbersome and relatively insensitive. Recently, array-based methods have been developed to measure gene expression simultaneously for large numbers of genes. The method involved spotting hundreds or thousands of cDNAs on either glass slides or filters. The method can provide quantitative measurement of the expression levels of thousands of genes in different tissues.

Although studies using cDNA arrays have been carried out to investigate drug metabolizing enzyme mRNA levels in liver, it is not known if similar information can be obtained from blood samples. A blood-based method would eliminate the need for liver biopsies to determine drug-metabolizing enzyme levels and to predict drug clearance in individuals.

### SUMMARY OF THE INVENTION

The present invention provides a blood based method of predicting the clearance of drugs. The method comprises using DNA arrays for detecting mRNAs for multiple CYPs, drug metabolizing enzymes, and transporters in blood collectively termed here as “drug clearance markers”.

The RNA materials can be prepared from a single peripheral blood sample. The RNA prepared from blood cells is reverse transcribed to form cDNAs which are then hybridized to the DNA arrays. The DNA arrays comprise discrete spots of specific nucleotide sequences for desired genes. Positive signals are taken as indicators of the presence of mRNAs. The presence of mRNAs in blood cells is correlated to the presence of mRNAs in liver, which in turn is correlated to the protein levels. From the data on protein level (or RNAs in blood cells or liver), unbound internal drug clearance can be estimated by standard statistical models. This can then be used to determine individualized drug dosage.

### BRIEF DESCRIPTION OF THE DRAWINGS

FIG. 1A is a representation of the raw image obtained from a phosphorimager from a human peripheral blood mononuclear cell RNA-derived sample following processing on a DNA array.

FIG. 1B is the processed image of FIG. 1A indicating that each filter contains two fields numbered 1 and 2 and each field is divided into 8 grids labeled from right to left as A-H, and each grid is organized into 30 rows and 12 columns.

FIG. 2A is a representation of the correlation of the image intensity measured by the phosphorimager (DLU/mm²) versus the corresponding amount of radioactivity indicating linearity between the phosphoimager count and the amount of radioactivity on each standard with a correlation coefficient of 0.98 and the slope (1.8×10⁵±1068.8).

FIG. 2B is a representation of the correlation between each area obtained from phosphoimager of the standard 1 and the counts for the area on standard 2 with r value of 0.99.

FIG. 3 is a plot of the normalized intensities from the two independent sets of analyses of the same phophorimage indicating a correlation coefficient of r=0.99. The plot shows that the data obtained by the method of the present invention is reproducible.

FIG. 4 is a plot of the normalized intensity of the housekeeping genes in field 1 against the corresponding intensities of the same genes in field 2 for a representative peripheral blood cell-derived mRNA sample. This plot shows the intra-filter reproducibility of the results obtained by the method of the present invention.

FIG. 5 is a plot of normalized intensities from two hybridizations and is an indication of the inter-filter reproducibility. The mean ratio of normalized intensity from the two hybridizations was 1.04±0.296. For mRNAs with normalized intensity of 1000 or greater, the level of the variability is better (mean ratio of 0.94±0.18)

FIG. 6 is a representation of the mean concentration of the CYP in human hepatic microsomes from (Shimada et al., 1994, J. Pharmacol. Exp. Therap., 270:414-423) versus the mean normalized intensity of the same CYPs in peripheral blood mononuclear cells obtained using DNA arrays. The solid line represents the best fit line through the mean values.

FIGS. 7A-C are representations of the levels of CYPs (FIG. 7A), drug metabolizing enzyme Glutathione transferase (FIG. 7B) and transporters (FIG. 7C) in human hepatic microsomes versus the means normalized intensity of the same CYPs in the peripheral blood cells according to the present invention.

FIG. 8 is a representation of the mean activity of the phase II drug metabolizing enzymes in human liver fractions from Iyer and Sinz, (1999, Chem. Biol. Interac., 118:151-169) versus the mean normalized intensity of the spots corresponding to the same activities in peripheral blood mononuclear cells obtained using DNA arrays of the present invention. The solid line represents the best fit line through the mean values.

FIG. 9 is a representation of the results using reverse transcriptase-polymerase chain reaction (RT-PCR). Three samples of patients with multiple sclerosis (Lanes P1, P2, P3) and 3 control samples (Lanes C1, C2, C3) were analyzed using primers specific for actin (FIG. 9A), CYP 2E1 (FIG. 9B), CYP 1B1 (FIG. 9C), CYP 1A1 (FIG. 9D) and CYP 2B6 (FIG. 9E). A band corresponding to P3 was clearly noted in the gel for FIG. 9E but is not evident in this image. The arrowheads mark the fragment corresponding to the CYP. The lane marked M is a molecular weight marker containing 100 base pair ladder and the lines to the side of each gel align with 100 base pair, 500 base pair and 1000 base pair markers.

### DETAILED DESCRIPTION OF THE INVENTION

The present invention provides a method for detecting blood mRNA levels for drug clearance markers including CYPs, drug metabolizing enzymes and transporters. The method comprises the steps of using DNA arrays for detecting mRNAs for multiple CYPs, drug metabolizing enzymes, and transporters in blood. From these levels, corresponding levels of the mRNAs and proteins in liver can be estimated and used for determining individual drug dosage.

“CYPs” as used herein means Cytochrome P-450 enzymes including, but not limited to those listed in Table I.

“Drug metabolizing enzymes” as used herein means enzymes known or suspected to be involved in the metabolism of drugs including, but not limited to, those listed in Table II.

“Transporters” as used herein means proteins known or suspected to be involved in the transport of drugs across cells including, but not limited to, those listed in Table III.

Blood can be collected from an individual by any standard means. For example, peripheral blood can be obtained from individuals by venipuncture. It is preferable to add an anticoagulant such as heparin. The blood samples are then processed to isolate mononuclear cells. For example, the samples maybe centrifuged to form and isolate a buffy coat. The methodologies for this are well known to those skilled in the art. The buffy coat can then be subjected to density gradient centrifugation to isolate mononuclear cells. The mononuclear cells are subjected to further purification techniques to yield the desired population. Once the cells have been isolated, RNA is prepared from them.

RNA is prepared by standard methods such as those described by Sambrook et al. In a preferred embodiment, RNA can be isolated from the purified mononuclear cells such as lymphocytes or monocytes using RNA isolation reagents (such as the TRI reagent from Molecular Research Center, Inc. Cincinnati, Ohio). The TRI reagent is an improved version of the single step method of total RNA isolation. The reagent combines phenol and guanidine thiocyanate in a mono-phase solution to inhibit RNase activity. This method allows processing of a large number of samples for the isolation of total RNA or the simultaneous isolation of RNA, DNA and proteins. The entire procedure can be completed in about 1 hour with significant recovery of undegraded mRNAs. Purified RNA can be stored at −80° C. or used immediately. Total RNA as purified above can be used as such or mRNA can be prepared therefrom by subjecting it to an oligo dT column.

The DNA array technology is used to determine the presence of relevant mRNAs in the RNA population purified from the blood cells. DNA arrays are now commercially available. DNA arrays can provide gene expression profiles of cells from desired tissues. One example is GeneFilter GF211, containing 5188 spots of named human genes with 5 ng of an approximately 1000 base long, 5′ end-derived PCR fragment on each spot. Hybridization of the DNA filters is carried out by standard procedures. In general, filters may be prewashed to reduce non-specific binding. The purified RNA or mRNA isolated from blood samples is labeled. Any type of label including fluorescent, enzyme-based or radioactive labels may be used. It is preferable to use radioactively labeled probes because of their high sensitivity. Hybridizations are carried out under stringent conditions. Unbound materials are washed away and array spots having positive signals are detected. The image is captured and processes using automated image processors.

Image analysis of the TIFF images can be carried out by commercially available software. Typically the software adjusts the image quality based on positive controls. Further, orientation of the image is also facilitated by positive controls. It is desirable to create a grid that locates each spot, analyze the image corresponding to each spot, map the image information to the identity of the gene, and input the information into a database. An example of a suitable software is Image Analysis Software Pathways (Research Genetics, Inc.). The software locates, calculates, and stores each cDNA spot intensity from each TIFF file and simultaneously compares two different normalized TIFF images.

The data from the captured images is subjected to data analysis to determine the statistical significance by using standard software such as SPSS 6.0 (SPSS Inc., Chicago, Ill.) and Excel (Microsoft Corp., Bellevue, Wash.).

From the mRNA levels determined as described above, an unbound hepatic intrinsic clearance (Cl_{u,int}) which can be scaled up to hepatic clearance using well established hepatic clearance models that incorporate protein binding and liver blood flow and mixing patterns.

The following examples are presented to further describe the invention and are intended to be illustrative and not restrictive.

### EXAMPLE 1

This embodiment describes the method used in the present invention and demonstrates that the present method is reproducible and reliable. Peripheral blood anticoagulated with heparin was obtained by venipuncture. Within 4 hours of collection, the blood samples centrifuged at 200×g for 10 minutes with a swinging bucket rotor and a buffy coat was isolated. The buffy coat was diluted with 2 volumes of phosphate buffered saline (PBS) and overlaid on a cushion of Hypaque-Ficoll (Histopaque, Sigma Chemical, St. Louis, Mo.). Density gradient centrifugation at 900-1000 g for 30 minutes on the swinging bucket rotor yielded mononuclear cells. The cells were washed 3 times with PBS and resuspended in RPMI-1640 medium containing 10% v/v fetal bovine serum. The cell suspension was incubated at 37° C. for 2 hours in cell culture flasks to deplete monocytes. The monocytes adhered to the flask and the supernatant yielded a mononuclear cell population that was enriched in lymphocytes. The adherent monocytes were gently rinsed with 5 ml of RPMI-1640 medium containing 10% v/v fetal bovine serum to increase lymphocyte yield.

RNA was isolated from the purified lymphocytes and monocytes using the TRI reagent (Molecular Research Center, Inc. Cincinnati, Ohio). The cells (5×10⁶ cells for lymphocytes or a 10 cm² area of culture plate for monocytes) were lysed in 1 ml TRI reagent and the homogenate was separated into aqueous and organic phases by the addition of bromochloropropane (0.1 ml) or chloroform (0.2 ml) and centrifugation. RNA remained exclusively in the aqueous phase, DNA in the interphase, and proteins in the organic phase. RNA was precipitated from the aqueous phase by addition of 0.5 ml isopropanol, washed with 1 ml of 75% v/v ethanol and solubilized in twice autoclaved diethyl pyrocarbonate treated water. Samples were stored at −80° C.

The DNA array filters were prewashed in 0.5% SDS to rid the filter of any residues since this results in cleaner hybridizations with less background noise 0.5% SDS solution was heated until boiling, the boiling solution was poured over membranes and gently agitated for five minutes.

Total RNA concentrations were measured using a spectrophotometer. The total RNA was diluted 1:200 in phosphate buffered saline and the absorbance spectrum between 220-400 nm was obtained. The RNA concentrations were obtained using a conversion factor of 44 μg per unit of optical density at 260 nm. The ratio of absorbance at 260 nm to absorbance at 280 nm was computed because values in the range 1.8-2.0 are indicative of purity.

Hybridizations can be carried out with 40 ng of mRNA or 1-5 μg of total RNA. Lower amounts of mRNA yielded an acceptable signal but needed more exposure time. Higher amounts of mRNA (more than 200 ng) could be used with faster exposure times or to measure the expression levels of less abundant mRNAs.

For each labeling, 5 μg of total RNA was reverse-transcribed in the presence of 100 μCi of ³²P dCTP (ICN Radiochemicals), 2 μg of Oligo-dT, 1.5 μl of dNTP mixture containing dATP, dGTP, and dTTP (Pharmacia), 1.0 μl of DTT (Life Technologies) and 200 units of SuperScript II RT (Life Technologies, Inc.). The labeled cDNA was denatured and hydridized to the cDNA GeneFilter GF211 which contain named human genes Research Genetics Inc. (Huntsville, Ala.). GeneFilters contain 5188 spots each with 5 ng of an approximately 1000 base long, 5′ end-derived PCR fragments. The GeneFilters were prehydridized at 42° C. in a roller oven with 1.0 μg/ml poly-dA (Research Genetics, Inc, Hunstville, Ala.) and 1.0 μg/ml Cot1 DNA (Life Technologies, Inc.) in 5 ml of Microhyb solution (Research Genetics, Inc.) for at least 2 hours. After hybridization, the filters were washed twice at 50° C. in 2×SSC (1μ SSC, 15 mM trisodium citrate, and 150 mM NaCl), 1% SDS for 20 minutes and once at room temperature in 0.5μ SSC, 1% SDS for 15 minutes. The filters were then exposed overnight to a Packard high resolution phosphor screen and scanned at 50 μm resolution in a Cyclone phosphorimager (Packard Instrument, Meriden, Conn.). After each hydridization, the filters were stripped by boiling in 0.5% SDS solution and scanned for residual leftover hybridization.

TIFF images resulting from the phosphoimager were directly imported by using the image analysis software Pathways (Research Genetics, Inc.). In each grid, the first and second columns contained multiple total genomic DNA positive control spots. This pattern of the control spots helped to orient the filters for the image processing software and to monitor the homogeneity of the hybridization. Importantly, these control spots could be used to align the images and for autocentering. Upon successful alignment the software program: i) created a grid that could locate each spot, ii) analyzed the image corresponding to the spots, iii) mapped the image information to the identity of the gene, and iv) updated a database with the information.

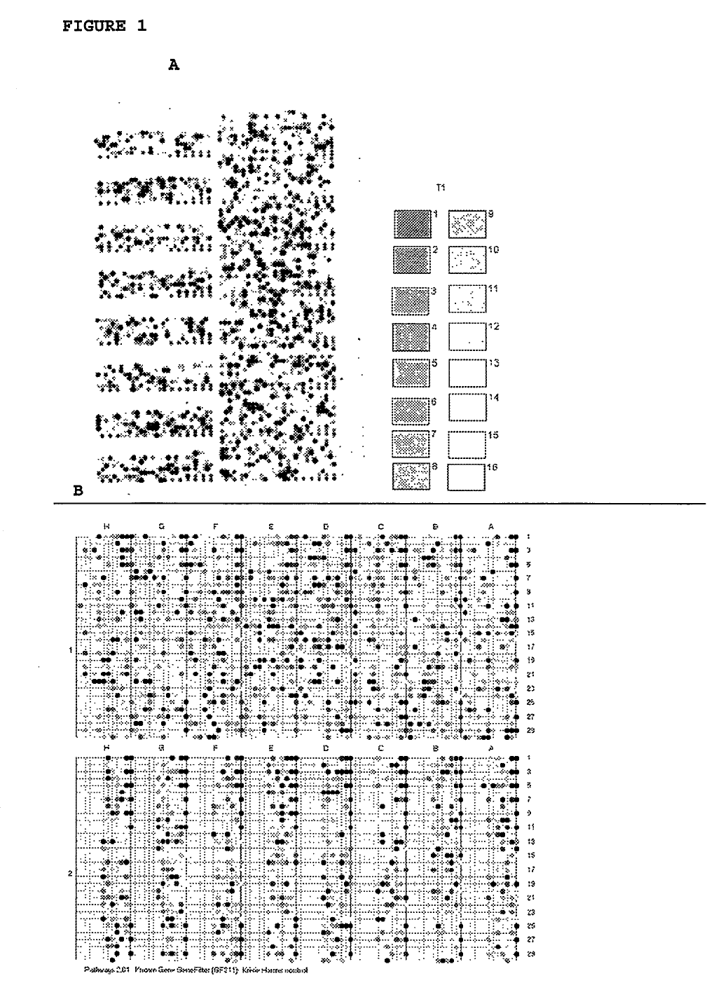

An example of a raw image obtained from the phosphorimager after processing with a representative human peripheral blood mononuclear cell RNA-derived sample is shown in FIG. 1A. Each spot on the array contains a known cDNA and immoblizes a single labeled cDNA from the sample. The intensity of the spot thus corresponds to the expression of a known mRNA. The rectangular areas on the image correspond to a filter containing ¹⁴C standards that were included during exposure of the Genefilter on the phosphormager to provide a control for spatial homogeneity and to allow absolute referencing of filters if needed. The raw image was imported into Pathways software, rotated, aligned and autocentered. This process allows the software to identify and quantitate the spot intensity. In addition, the software links each spot to the cDNA and creates a database containing information regarding the experiment, the filter, spot intensity and gene identities.

The processed image (FIG. 1B) shows that each filter contains two fields numbered 1 and 2. Each field is divided into 8 grids labeled from right to left as A-H, and each grid is organized into 30 rows and 12 columns. In each grid, the first and second columns contain multiple control total genomic DNA positive control spots. This pattern of the control spots help to orient the filters for the image processing software and to monitor the homogeneity of the hybridization. The GF211 GeneFilters also contains multiple housekeeping genes that did not differ in hybridization signal between several different tissues in an analysis conducted at the National Institute of Health by two-color fluorescence. Some of these housekeeping gene names are listed in Appendix 1. These genes are not necessarily expressed at the same level in the same or different tissues, i.e., some seem to be expressed at very low levels per cell while others seem to be expressed at much higher levels.

A three-pronged approach was used for data analysis. In the first step, statistical analysis was conducted using software such as SPSS 6.0 (SPSS Inc., Chicago, Ill.) and Excel (Microsoft Corp., Bellevue, Wash.) for specific categories of genes such as i) cytokines and cytokine receptors, ii) adhesion molecules, and iii) immunological molecules involved in antigen presentation and signaling.

The normalized intensity data was exported to a statistical software program, such as SPSS and appropriate ANOVAs and post hoc t-tests were used to arrive at decisions regarding statistical significance. The inclusion the ¹⁴C internal standard allowed assessment of the linearity of the imaging process because the amount of radioactivity in each of the 16 rectangles of the standard is known.

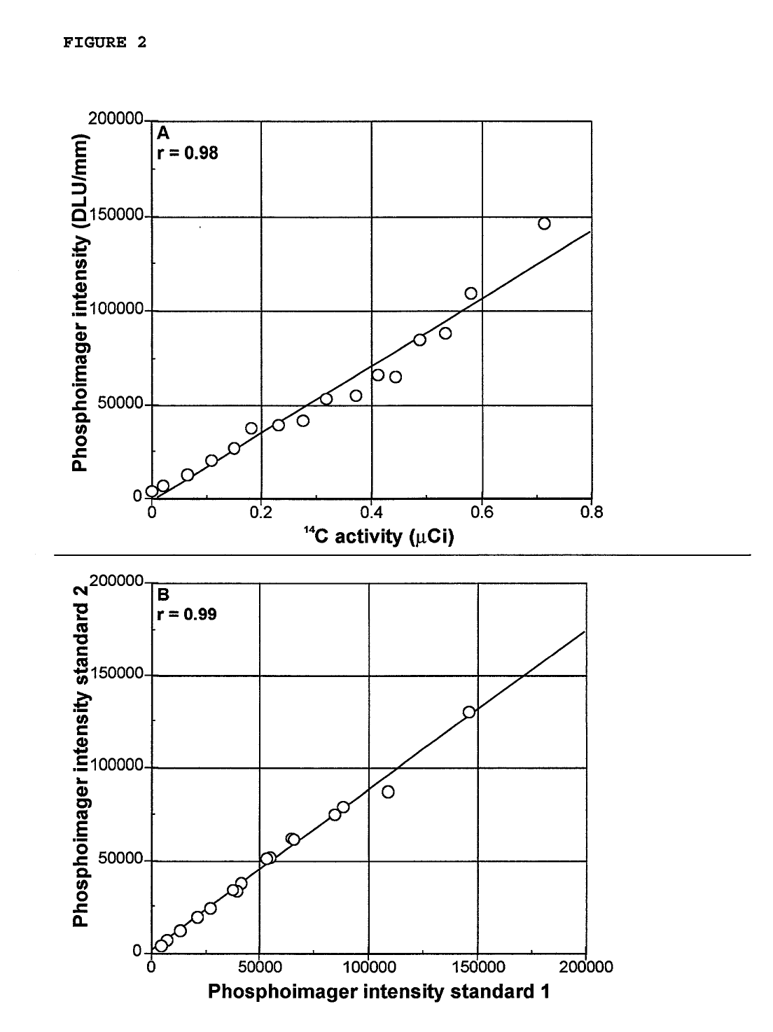

FIG. 2A plots the image intensity measured by the phosphorimager versus the corresponding amount of radioactivity of the linearity between the phosphoimager count (DLU/mm²) and the amount of radioactivity on each standard. The correlation coefficient was r=0.98 and the slope (1.8×10⁵±1068.8). This demonstrates that phosphorimages can be used instead of radioactivity measurements.

The reproducibility of two ¹⁴C internal standards on the same scan was also determined. FIG. 2B shows the counts of each area obtained from phosphoimager analysis of standard 1 was highly correlated with the counts for the area of standard 2 with an r value of 0.99. The reproducibility of data analysis was verified because the GeneFilters DNA array contains over 5000 spots and small errors in the alignment can result in substantive errors in mRNA identification.

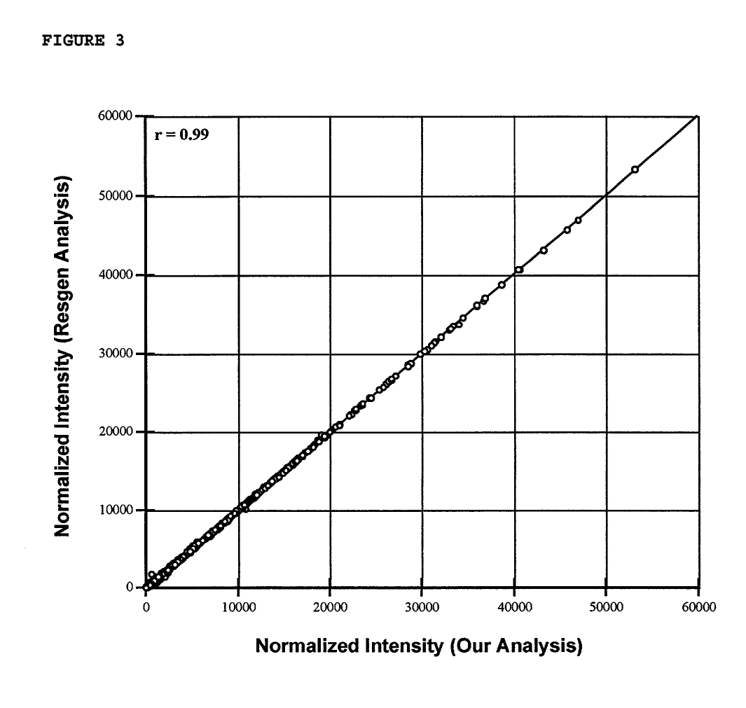

To cross-check/verify the data analysis procedure, independent analysis of an image at two different facilities was conducted. The results, shown in FIG. 3, plot the normalized intensities from the two independent sets of analyses. The plot shows a strong correlation with r=0.99. The slope of the regression curve was 1.00±0.0002.

The GF211 Genefilter contains several housekeeping genes that are spotted in duplicate in each field of the Filter allowing assessment of intra-filter spotting and spatial heterogeneity in hybridization efficiency.

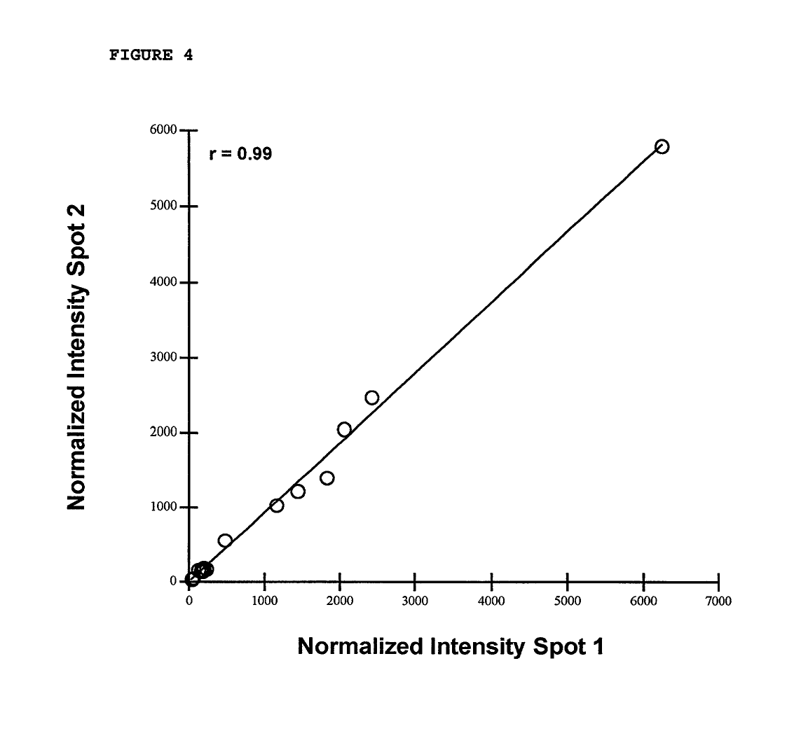

FIG. 4 is a plot of the normalized intensity of the housekeeping genes in field 1 against the corresponding intensities of the same genes in field 2 for a representative peripheral blood cell-derived mRNA sample.

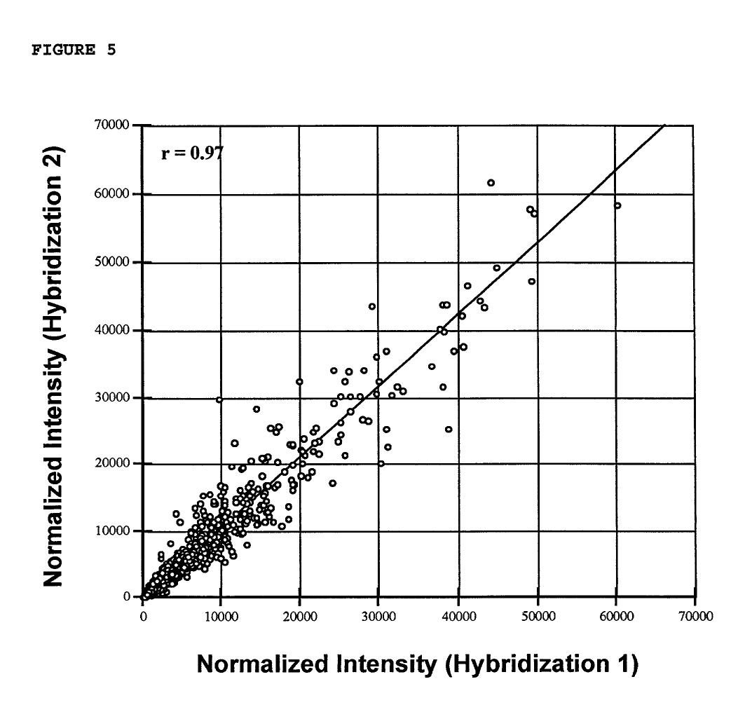

To examine inter-filter variability, we independently labeled (reverse transcribed) the same mRNA sample and hybridized it. The results are summarized in FIG. 5, which plots the normalized intensities from each hybridization. The mean ratio of normalized intensity from the two hybridizations was 1.04±0.296. For mRNAs with normalized intensity of 1000 or greater, the level of the variability is better (mean ratio of 0.94±0.18)

### EXAMPLE 2

This embodiment demonstrates that blood cell RNA for CYPs, drug metabolizing enzymes and transporters can be detected in blood cells.

Cytochrome P-450 Expression

The normalized intensity of drug metabolizing CYP mRNA expression in peripheral blood in the samples is summarized in Table 1. The signals corresponding to CYP 4A11, CYP 2J2 and CYP2E1 were strong while the other CYPs were weaker by comparison. The coefficient of variation ranged from 18% for CYP 2J2 to 114% for CYP 2A6. The results demonstrate the sensitivity of the method and the feasibility of detecting the expression of multiple CYPs from single peripheral blood samples.

TABLE 1
Signals (arbitrary units obtained from the DNA
arrays for the various cytochrome P-450 (CYP) enzymes.
The coefficient of variation (CV) ranges from 18 to 114%.

(n = 10)

| Gene | Mean | CV % |
|---|---|---|
| CYP 4A11 | 6.5 × 10³ | 22 |
| CYP 2J2 | 2.1 × 10³ | 18 |
| CYP 2E1 | 1.4 × 10³ | 75 |
| CYP 27 | 1.1 × 10³ | 62 |
| CYP 21 | 9.0 × 10² | 66 |
| CYP 2A6 | 8.2 × 10² | 114 |
| CYP 1A1 | 6.3 × 10² | 47 |
| CYP 2B6 | 4.1 × 10² | 55 |
| CYP 4B1 | 4.1 × 10² | 89 |
| CYP 27 | 3.5 × 10² | 46 |
| CYP 17 | 3.5 × 10² | 36 |
| CYP 2C8 | 2.7 × 10² | 70 |
| CYP 3A5 | 2.7 × 10² | 30 |
| CYP 1B1 | 3.6 × 10² | 45 |
| CYP 2C9 | 3.2 × 10² | 58 |
| CYP 19 | 1.3 × 10² | 22 |

Expression of Other Drug Metabolizing Enzymes

The signals corresponding to several drug metabolizing enzymes are summarized in Table II. The uridine diphosphate glucuronosyl transferases (UGTs) are an important class of Phase II conjugating enzyme. Probes corresponding to three isozymes, UGT 2B4, UGT 2B10 and UGT 2B15 were available on the array. The strongest signal corresponded to UGT 2B10.

The range of other transferase enzymes such as catecholamine-O-methyltransferase (COMT), thiopurine S-methyltransferase, and the steroid sulfotransferases, DHEA-preferring sulfotransferase and hydroxysteroid sulfotransferase were also determined. Robust signals corresponding to COMT and the two steroid sulfotransferases were detected (Table II).

Several glutathione-S-transferase (GST) probes were also immobilized on the DNA array and we were able to examine the expression of several isozymes (Table II). The signals corresponding to GST M4, GST A3 and a GST homolog were particularly notable. The GST signal strengths ranged over a greater than 200-fold range of intensity depending on the isozyme. These findings demonstrate that support the premise that considerable inter-isozyme selectivity is obtained.

TABLE 2
Signals (arbitrary units) obtained from the DNA
arrays for various drug metabolizing enzymes.

(n = 10)

| | Gene | Mean | CV % |
|---|---|---|---|
| UGT 2B4 precursor, | UGT 2B4 | 1.4 × 10² | 28 |
| microsomal | | | |
| UGT 2B10 precursor, | | 1.3 × 10³ | 23 |
| microsomal | | | |
| UCT 2B15 precursor | UGT2B15 | 1.1 × 10³ | 52 |
| Catechol-O- | COMT | 2.4 × 10³ | 149 |
| methyltransferase | | | |
| Thiopurine 5- | TPMT | 2.6 × 10² | 44 |
| methyltransferase | | | |
| Hydroxysteroid | HSST2 | 1.1 × 10³ | 70 |
| sulfotransferase | | | |
| DHEA-preferring | STD | 2.2 × 10³ | 76 |
| sulfotransferase | | | |
| GST M2 | GSTM2 | 6.2 × 10² | 45 |
| GST theta 2 | GSTT2 | 4.9 × 10² | 29 |
| GST M4 | GSTM4 | 1.3 × 10⁴ | 10 |
| GST M3 | GSTM3 | 4.7 × 10² | 24 |
| GST MS | GSTM5 | 2.4 × 10² | 36 |
| GST pi-1 | GSTP1 | 1.8 × 10² | 12 |
| GST A2 | GSTA2 | 4.5 × 10² | 11 |
| GST theta 1 | | 1.1 × 10³ | 50 |
| GST A3 | GSTA3 | 1.2 × 10³ | 101 |
| Microsomal GST | | 1.8 × 10² | 31 |
| Microsomal GST 2 | MGST2 | 2.0 × 10² | 49 |
| Microsomal GST 3 | MGST3 | 7.3 × 10² | 48 |
| GST homolog | | 1.3 × 10⁴ | 71 |

Abbreviations:
UGT: UDP glucuronosyl transferase; DHEA: Dihydroepiandrosterone; GST: Glutathione S-transferase.

Expression of Transporters

Since transporter activity contributes significantly to the clearance of many drugs as well to the emergence of drug resistance, the expression of transporter mRNAs was examined. Probes corresponding to 5 proteins, MDR1, MDR3, MRP1, MRP3 and MRP5, that have been linked to multi-drug resistance were tested. A strong signal for MRP1 was consistently detected.

Probes corresponding to wide range of transporters were available on the DNA array used and only a subset of these are presented in Table III. The signals corresponding to several transporters of potential pharmaceutical interest; e.g., transporters involved in creatinine, betaine, monocarboxylic acid and nucleoside transport were readily and consistently detected.

TABLE 3
Signals (arbitrary units) obtained from the DNA
arrays for the various transporters.

| | | (n = 10) | |
|---|---|---|---|
| | Gene | Mean | CV % |
| MDR 1 | | 2.9 × 10² | 28 |
| MDR 3 | PGY3 | 3.6 × 10² | 50 |
| MDR-associated protein 1 | MRP1 | 5.2 × 10³ | 33 |
| MDR-associated protein | MRP3 | 3.6 × 10² | 76 |
| homolog-3 | | | |
| MDR-associated protein | MRP5 | 1.0 × 10² | 33 |
| homolog-5 | | | |
| Creatine transporter | | 1.2 × 10⁴ | 45 |
| NBMPR-insensitive | ENT2 | 1.8 × 10³ | 14 |
| nucleoside transporter | | | |
| X-linked PEST-containing | XPCT | 5.4 × 10³ | 24 |
| transporter | | | |
| Neutral amino acid | | 4.0 × 10³ | 61 |
| transporter B | | | |
| Monocarboxylic acid | SLC16A1 | 3.6 × 10³ | 15 |
| transporter | | | |
| Putative monocarboxylate | MCT | 2.3 × 10³ | 82 |
| transporter | | | |
| Na/Cl dependent betaine | | 2.1 × 10⁴ | 41 |
| transporter | | | |
| Amiloride sensitive | SLC9A1 | 4.8 × 10³ | 98 |
| Na+/H+ antiporter | | | |
| Tetracycline transporter- | | 1.0 × 10⁴ | 16 |
| like protein | | | |

Abbreviations:
MDR: Multidrug resistance; MRP: Multidrug resistance associated protein.

### EXAMPLE 3

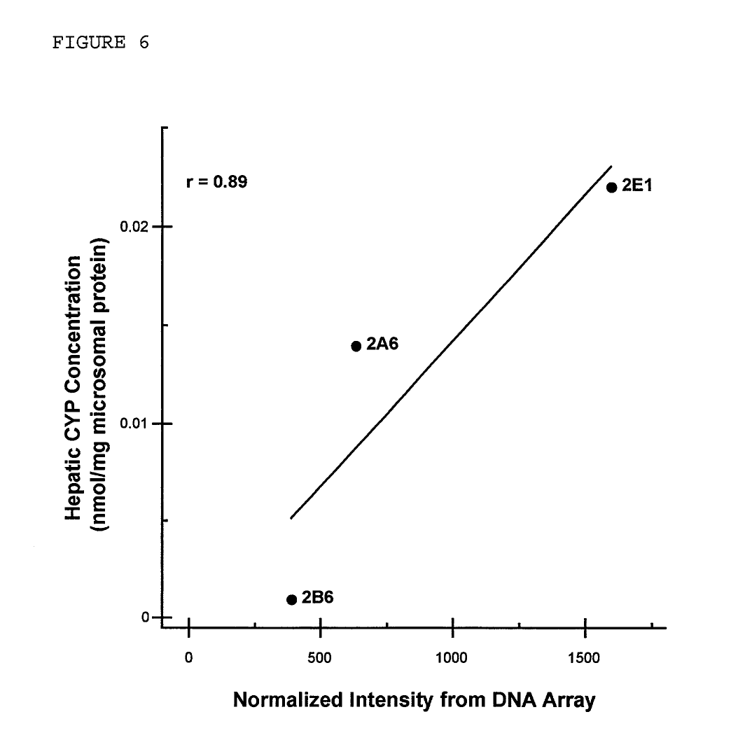

This embodiment demonstrates that the method of the present invention can be used to estimate liver CYP mRNA levels from the measured blood CYP mRNA levels. To illustrate this embodiment, the peripheral blood CYP expression levels obtained using DNA arrays were correlated to the values for CYP expression in the liver previously reported by others (Shimada et al., 1994) and also to the levels obtained by us. These authors examined the levels of CYPs 1A2, 2A6, 2B6, 2C, 2D6, 2E1 and 3A4 using immunochemical methods. The DNA arrays used provided corresponding mRNA levels for 2A6, 2B6, and 2E1. FIG. 6 plots the mean values for the normalized intensity of the CYP spots from the DNA array (n=20) against the immunochemical measure of CYP protein level from Shimada et al. (supra). The r value of the linear regression line was 0.89 and the correlation achieved a P value of 0.15. There was exact correspondence between the rank orders for the 3 CYPs among the two methods (Spearman r=1.00). This data supports the premise that DNA arrays measurement in peripheral blood can be used for estimating CYP levels in liver.

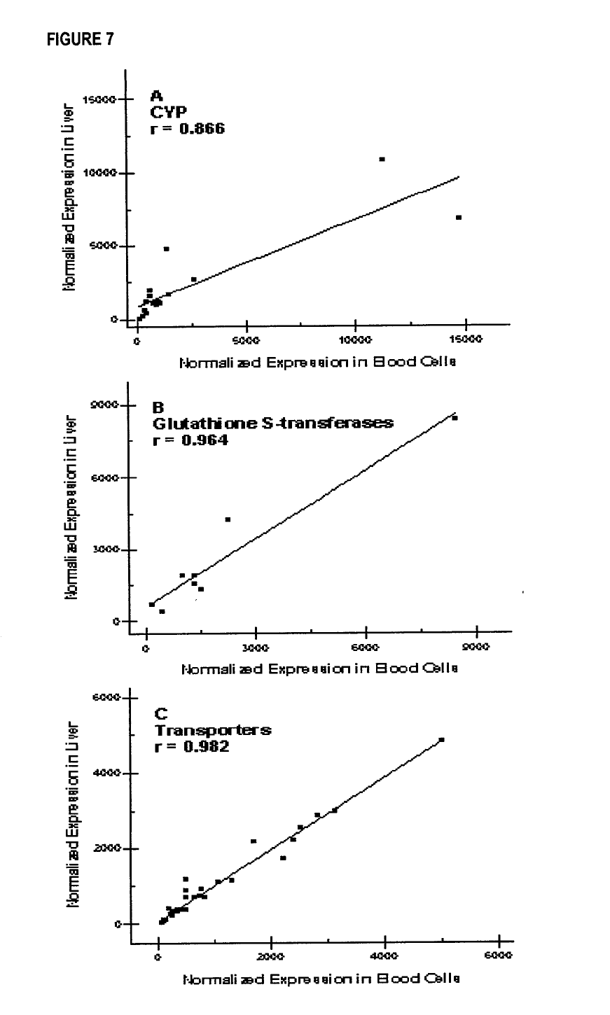

In another illustration of this embodiment, the relationship between mRNA levels from blood cells and liver mRNA levels was determined. The results shown in FIG. 7A indicate a strong correlation between the two.

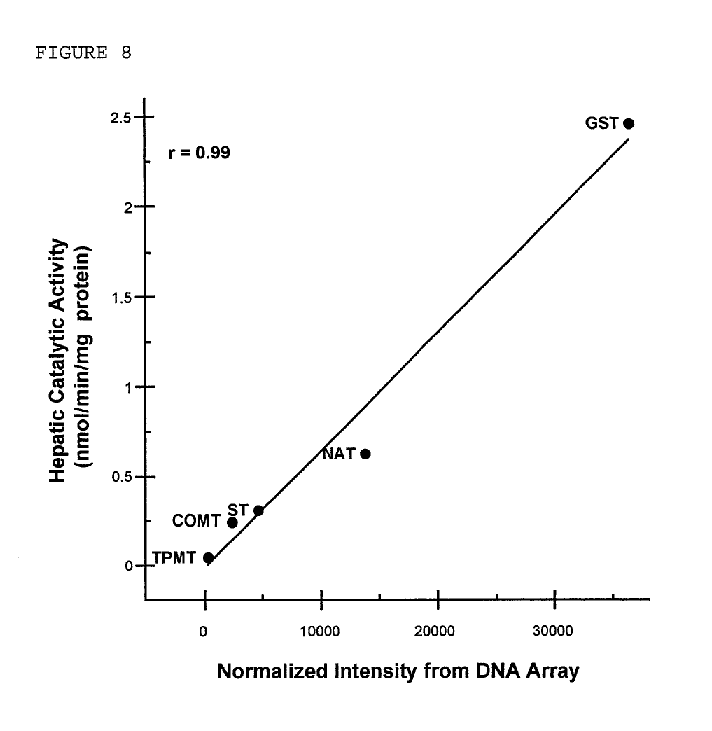

The level of Phase II enzymes levels from DNA arrays was compared with the activities of 5 Phase II enzymes reported by Iyer and Sinz (1999) in human liver fractions. The enzymes compared were: glutathione S-transferase (GST), UDP glycosyltransferase, sulfotransferase (ST), N-acetyl transferase (NAT), thiopurine methyl transferase (TPMT), and catechol O-methyl transferase (COMT). The normalized signals of the spots on the DNA array corresponding to these activities (the extracellular matrix sulfotransferases were excluded from the analysis for ST; protein N-acetyl transferases were excluded for NAT) and plotted the data against the activities reported by Iyer and Sinz (FIG. 8). The UDP glucuronsyl transferase activities did not correlate between the two methods but the r value for the regression line for the 5 remaining enzymes was excellent (r=0.99) and the correlation achieved a P value of 0.002. The rank order correspondence for these 5 Phase II enzymes was exact (Spearman r=1.00).

In another illustration of this embodiment, the levels of mRNA in blood for the Phase II enzymes were compared with the levels determined in liver. As shown in FIG. 7B a strong correlation is observed.

In yet another illustration of this embodiment, the levels of mRNA in blood cells for transporters were compared with the levels determined in liver. As shown in FIG. 7C, a strong correlation is observed.

### EXAMPLE 4

This embodiment confirms the presence of several CYPs mRNAs in a representative subset of 3 mRNA preparations. The reverse transcription conditions were similar to those used for labeling mRNA for DNA arrays except that ³²P CTP was not included. The PCR conditions were derived from (Baron et al., 1998). The following splice junction spanning primers were used to minimize the amplification of any contaminating genomic DNA (Baron et al., 1998). The sense (S) and antisense primers (AS) were: CYP 1B1-S, 5′-GTA TAT TGT TGA AGA GAC AC-3′ (SEQ ID NO:1) and CYP 1B1-AS, AAA GAG GTA CAA CAT CAC CT-3′ (SEQ ID NO:2), 316 base pair product; 2E1-S 5′-AGC ACA ACT CTG AGA TAT GG-3′ (SEQ ID NO:3) and CYP 2E1-AS 5′-ATA GTC ACT GTA CTT GAA CT-3′ (SEQ ID NO:4), 366 base pair product; 2B6/7-S 5′-CCA TAC ACA GAG GCA GTC AT-3′ (SEQ ID NO:5) and CYP 2B6/7-AS 5′-GGT GTC AGA TCG ATG TCT TC-3′ (SEQ ID NO:6), 377 base pair product; -Actin-S, 5′-ACC CAC ACT GTG CCC ATC TA-3′ (SEQ ID NO:7) and -Actin-S, 5′-CGG AAC CGC TCA TTG CC-3′ (SEQ ID NO:8), 290 base pair product. The PCR conditions were 35 cycles with 1 minute of annealing at 56° C., 2 minutes of extension at 72° C. and 1 minute of denaturation at 93° C., with 5 minutes extension. The PCR products were separated on 1% agarose gels. The amplification of CYP 1A1 was according to Vanden Heuval et al. (1993, Carcinogenesis, 14:2003-2006). The primers were CYP 1A1-S, 5′-TAG ACA CTG ATC TGG CTG CAG-3′ (SEQ ID NO:9) and CYP 1A1-AS, 5′-GGG AAG GCT CCA TCA GCA TC-3′ (SEQ ID NO:10), 148 base pair product. The PCR conditions were 30 cycles with 30 sec of annealing at 54° C., 1 minute of extension at 72° C. and 15 seconds of denaturation at 94° C. The PCR products were separated on 3% agarose gels.

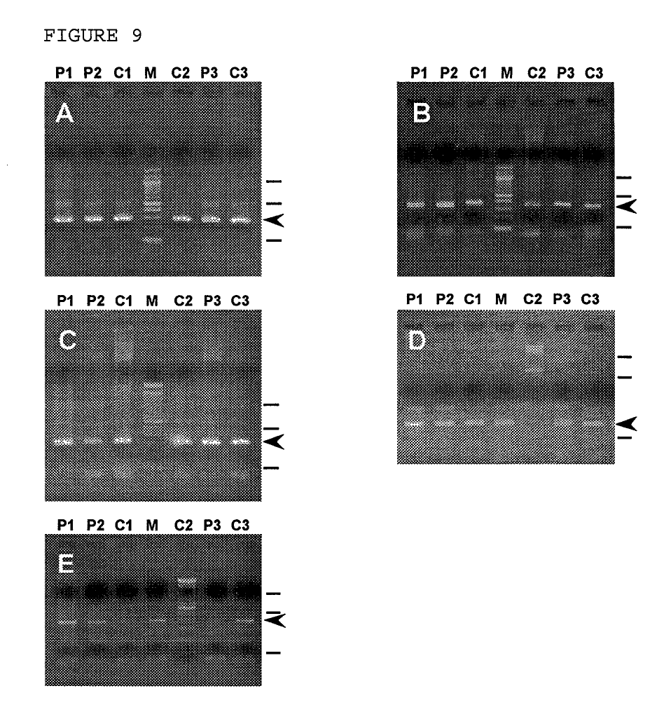

FIG. 9 shows the PCR products using primer pairs for CYPs 2E1, 1B1, 1A1, 2B6/7 and actin. For each of these CYPs, products of the expected length were observed demonstrating that the mRNA is present in each sample examined.

What is claimed is:

1. A method of detecting in an individual the levels of mRNAs for drug clearance markers selected from the group consisting of CYPs, drug metabolizing enzymes and transporters comprising the steps of:
a. obtaining a blood sample from the individual;
b. isolating cells from the sample;
c. preparing total RNA from the cells;
d. reverse transcribing the mRNAs in the total RNA in c. to produce cDNAs;
e. detecting the level of reverse transcribed cDNAs by hybridizing said cDNAs to specific probes for said markers; and
f. determining the level of corresponding mRNAs from the reverse transcribed cDNA levels.

2. The method of claim 1 further comprising the step of isolating mRNA from the total RNA prior to determining the presence of desired mRNAs in step d.

3. The method of claim 1 further comprising the step of correlating the levels of blood mRNAs to the levels of liver mRNAs.

4. The method of claim 3, further comprising the step of correlating the levels of liver mRNAs to liver protein levels for the desired mRNAs.

5. The method of claim 1, wherein the hybridization is carried out using a DNA array.

6. The method of claim 1, wherein the CYPs are selected from the group consisting of CYP 4A11, CYP 2J2, CYP 2E1, CYP 27, CYP 21, CYP 2A6, CYP 1A1, CYP 2B6, CYP 4B1, CYP 27, CYP 17, CYP2C8, CYP 3A5, CYP 1B1, CYP 2C9, CYP 19.

7. The method of claim 1, wherein the drug metabolizing enzymes are selected from the group consisting of UDP glucuronosyl transferase, dihydroepiandrosterone, glutathione S-transferase, catechol-O-transferase, thiopurine S-methyltransferase and hydrozysteroid sulfotransferase.

8. The method of claim 1, wherein the transporters are selected from the group consisting of
MDR 1, MDR 3, MDR-associated protein 1, MDR-associated protein homolog-3, MDR-associated protein homolog-5, Creatine transporter, NBMPR-insensitive nucleoside transporter, X-linked PEST-containing transporter, Neutral amino acid transporter B, Monocarboxylic acid transporter, Putative monocarboxylate transporter, Na/Cl dependent betaine transporter, Amiloride sensitive Na+/H+ antiporter, and Tetracycline transporter-like protein.
